(19) United States
(12) Patent Application Publication
(10) Pub. No.: US 2020/0172605 A1
(43) Pub. Date: Jun. 4, 2020

(54) GENE THERAPY FOR ALZHEIMER'S AND OTHER NEURODEGENERATIVE DISEASES AND CONDITIONS

(71) Applicant: Cornell University, Ithaca, NY (US)

(72) Inventors: Ronald G. Crystal, New York, NY (US); Steven M. Paul, New York, NY (US)

(21) Appl. No.: 16/664,835

(22) Filed: Oct. 26, 2019

Related U.S. Application Data
(63) Continuation of application No. 14/911,400, filed on Feb. 10, 2016, now abandoned.
(60) Provisional application No. 61/874,118, filed on Sep. 5, 2013.

(51) Int. Cl. C07K 16/18; C12N 7/00; A61K 48/00; A61K 39/395

(52) U.S. Cl. CPC C07K 16/18; C12N 7/00; A61K 48/0075; A61K 48/0058; A61K 39/3955; A61K 2039/505; C07K 2317/76; C07K 2317/515; C07K 2317/51; C07K 2317/34; C12N 2750/14121

(57) ABSTRACT

Gene therapy compositions and methods to inhibit or treat neurodegenerative diseases, e.g., Alzheimer's disease, are provided.

### CROSS-REFERENCE TO RELATED APPLICATIONS

This application claims the benefit of the filing date of U.S. application Ser. No. 61/874,118, filed on Sep. 5, 2013, the disclosure of which is incorporated by reference herein.

### BACKGROUND

Alzheimer's disease (AD) directly affects 5.5 million Americans and is rapidly increasing n prevalence and economic impact. Existing drugs do little to limit the underlying disease process.

Extracellular plaques and intracellular neurofibrillary tangles are two pathological hallmarks of Alzheimer's disease. Plaques primarily consist of β-amyloid (Aβ) peptides, whereas tangles are composed of hyperphosphorylated and misfolded tau proteins. While the plaques vary in the tissues, tau pathology consistently displays a characteristic distribution pattern: the pathology originates in the entorhinal cortex (EC) and spreads to neighboring areas as the disease progresses (Braak H. and Braak E., 1991). This progressive tau pathology (neurofibrillary tangles and neurodegeneration) better correlates with the age-dependent decline of cognitive function in AD patients. It has been postulated that the progressive staging of tau pathology could result from either various degrees of vulnerability to tau pathology in different brain regions or spreading and transmission of pathologic (misfolded) tau within various neuronal networks. Recent evidence supports the spreading and transmission hypothesis, e.g., to explain the progressive staging of tau pathology. For example, Clavaguera et al. demonstrated that exogenous brain lysates containing misfolded tau can induce local tau aggregation, as well as the spread of local tau pathology throughout the brain of a recipient humanized tau mouse model without any existing tau pathology (Clavaguera et al., 2009). Consistent with this finding, Liu et al. and Calignon et al. found AT8- or MC1-positive misfolded or pathologic tau in areas other than EC in aged genetically-engineered mice which express a Frontal Temporal Dementia (FTD)-causing tau mutation (P301L)/protein restricted to the EC (Liu et al., 2012; Calignon et al., 2012). Importantly, brain regions outside the EC which contain AT8 and MCI-positive neurons do not contain mutant tau mRNA and are synaptically connected to EC. These results suggest that misfolded (or fibrillar) tau can be released into the extracellular space and be taken up (endocytosed) by adjacent neurons and further propagated, presumably via synapses. Indeed, such a trans-cellular propagation mechanism has also been demonstrated in an in vitro culture system (Frost et al., 2009; Kfoury et al., 2012) Furthermore, propagation/spreading of misfolded tau both in vivo and in vitro can be blocked by anti-tau antibodies, which sequester extracellular misfolded and fibrillar tau species.

These discoveries suggest that targeting pathological (but not wild-type) tau with an anti-tau monoclonal antibody could be a potential therapeutic strategy, in particular in situations that require reduction of misfolded tau species or to block the spread and propagation of misfolded tau. Of these, the targeting of hyper-phosphorylated tau epitopes by immunotherapy has emerged as a promising approach. For example, active immunization with a tau peptide (tau 379-408) containing phosphor-ser396 and -ser404 attenuates tau aggregation in brain and slows the progression of tangle-related pathology and behavior in 2 different types of tauopathy mouse models, P301L and htau/PS1M146V mice (Asuni et al., 2007; Boutajangout et al., 2010). Consistent with these findings, passive immunization with the PHF1 monoclonal antibody (recognizing p-ser396 and p-ser404) or the MC1 antibody (recognizing a conformational/pathological tau epitope) also reduces tau pathology and axonal degeneration, and improves behavioral deficits in P301S and P301L mice, (Boutajangout et al., 2011; Chai et al., 2012) again supporting that anti-tau immunotherapy may represent an intervention strategy for treating/preventing AD and (or) other tau-related diseases.

However, targeting pathological tau also requires treatment covering a large area of CNS. With passive immunization with anti-tau antibodies, the half-life of the anti-tau antibody requires weekly to monthly parenteral administration. Also, only a small portion of the anti-tau antibody administered into the blood stream (≤0.1%) will reach the CNS, where the antibody can capture the misfolded tau protein in the CNS extracellular space, consequently blocking the spread and propagation of tau pathology and reducing neurodegeneration

### SUMMARY

The invention provides compositions and gene therapy methods to treat, inhibit or prevent Alzheimer's disease (AD) and other neurodegenerative diseases and conditions, e.g., those associated with tau. In one embodiment, the invention provides an isolated nucleic acid sequence which encodes an antibody directed against tau. The antibody may recognize phosphorylated tau or a pathological conformation of tau, and in one embodiment includes sequences from monoclonal antibody MC1 or PHF1. The isolated nucleic acid sequence may encode an antibody fragment.

In one embodiment, the invention provides an isolated recombinant nucleic acid sequence comprises an open reading frame which encodes an antibody directed against tau, wherein the open reading frame comprises nucleic acid sequences for an Ig heavy chain specific for tau and nucleic acid sequences for an Ig light chain specific for tau. In one embodiment, the heavy and light chain sequences are from the same monoclonal antibody. In one embodiment, the isolated recombinant nucleic acid further comprises nucleic acid sequences for a protease cleavage recognition site interposed between the nucleic acid sequences for the Ig heavy chain and the nucleic acid sequences for the Ig light chain. In one embodiment, the open reading frame comprises sequences for an Ig heavy chain linked to sequences for a protease cleavage recognition site linked to sequences for an Ig light chain. In one embodiment, the open reading frame comprises sequences for an Ig light chain linked to sequences for a protease cleavage recognition site linked to sequences for an Ig heavy chain.

In one embodiment, the heavy chain is an IgG or IgM heavy chain. In one embodiment, the heavy chain is an IgG1, IgG2, IgG3 or IgG4 heavy chain. In one embodiment, the light chain is an Igk light chain. In one embodiment, the light chain is an Ig_{λ} light chain. In one embodiment, the Ig heavy chain nucleic acid sequences have at least 80%, 85%, 90%, 92%, 95%, 98% or 99% nucleic acid sequence identity to the heavy chain sequence in any one of SEQ ID No. 1, 2, 5, or 6. In one embodiment, the nucleic acid sequences for the Ig light chain have at least 80%, 85%, 90%, 92%, 95%, 98% or 99% nucleic acid sequence identity to the light chain sequence in any one of SEQ ID No. 1, 2, 5 or 6. In one embodiment, the Ig heavy chain has at least 80%, 85%, 90%, 92%, 95%, 98% or 99% amino acid sequence identity to the heavy chain sequence in SEQ ID No. 3 or 4. In one embodiment, the Ig light chain has at least 80%, 85%, 90%, 92%, 95%, 98% or 99% amino acid sequence identity to the light chain sequence in SEQ ID No. 3 or 4. I In one embodiment, the amino acid sequences encoded by the nucleic acid sequences for the Ig light chain or the Ig heavy chain that have at least 80%, 85%, 90%, 92%, 95%, 98% or 99% amino acid sequence identity to the heavy chain sequence in SEQ ID No. 3 or 4, may include both conservative and non-conservative substitutions. In one embodiment, the substitutions are conservative. In one embodiment, there are one or more conservative substitutions, e.g., 2, 5, 10, 20, or 30 (or any integer between 2 and 30) conservative substitutions. In one embodiment, there are one or more non-conservative substitutions, e.g., 2, 5, 10, 20, or 30 (or any integer between 2 and 30) non-conservative substitutions. In one embodiment, there are two or more substitutions, e.g., 2, 5, 10, 20, or 30 (or any integer between 2 and 30) substitutions. In one embodiment, the antibody recognizes phosphorylated Ser396, Ser404, Ser202, Ser262, Thr205, Ser356, Tyr394, or Tyr310. In one embodiment, the antibody recognizes a tau epitope comprising Ala2-Tyr18, Pro312-Glu342, Ser210-Ser241, Arg242-Lys281, Thr220-Ser235, or Arg230-Lys240. In one embodiment, the nucleic acid sequences encode an antibody fragment, e.g., Fv, Fab′ or scFv. In one embodiment, the open reading frame is operably linked to a promoter that is expressed in neurons, oligodendrocytes, glial cells or astrocytes.

The invention also provides a gene transfer vector comprising the isolated nucleic acid sequence which encodes an antibody directed against tau. In one embodiment, the nucleic acid may be driven by a cytomegalovirus/chicken beta-actin hybrid promoter or a glial fibrillary acidic protein promoter. The gene transfer vector may be an adeno-associated virus (AAV) vector, which may be selected from the group of AAVrh.10, AAV8 and AAV9 serotypes, or other viral vectors. For example, the invention provides, in one embodiment, a recombinant AAV or recombinant lentivirus comprising nucleic acid sequences encoding an Ig heavy chain of an anti-tau antibody, an Ig light chain of an anti-tau antibody, or an Ig heavy chain of an anti-tau antibody linked to an Ig light chain of an anti-tau antibody.

The invention further provides a composition comprising the gene transfer vector which in turn comprises the isolated nucleic acid sequence which encodes an antibody directed against tau, and a pharmaceutically acceptable carrier.

The invention provides a method of inhibiting or treating a neurodegenerative disease or condition characterized by pathological tau activity in a mammal, which may be a human, comprising administering the composition to the mammal. The disease or condition may be selected from the group comprising Alzheimer's disease, mild cognitive impairment, frontotemporal dementia, traumatic brain injury, stroke, transient ischemic attack, dementia, Creutzfeldt-Jakob disease, multiple sclerosis, prion disease, Pick's disease, corticobasal degeneration, Parkinson's disease, Lewy body dementia, Progressive supranuclear palsy; Dementia pugilistica (chronic traumatic encephalopathy); frontotemporal dementia and parkinsonism linked to chromosome 17; Lytico-Bodig disease; Tangle-predominant dementia; Ganglioglioma and gangliocytoma; Meningioangiomatosis; Subacute sclerosing panencephalitis; lead encephalopathy, tuberous sclerosis, Hallervorden-Spatz disease, and lipofuscinosis; Argyrophilic grain disease; and Frontotemporal lobar degeneration. In one embodiment, the amount of the composition that is administered is effective to decrease tau pathology, e.g., decrease tangle development, decrease soluble tau or decrease insoluble tau in the brain, improve motor performance, e.g., balance or coordination, and/or improve cognitive function. The effect is sustained over weeks, months or years.

In one embodiment, the mammal is a human. In one embodiment, the composition is administered intracranially, intraventicularly, or intracisternally. In one embodiment, the composition is administered to the hippocampus or entorhinal cortex. In one embodiment, the human has an ApoE4 allele.

### BRIEF DESCRIPTION OF THE FIGURES

FIG. 1. Diagram of the PHF-1 and MC1 gene design and expression cassette. The AAVrh.10MC1 and AAVrh.10PHF-1 vectors have the AAVrh.10 capsid and the MC1 or PHF-1 expression cassette flanked by two inverted terminal repeats (ITR). A). Schematic of the full length antibody including light and heavy chains. B). Diagram of the AAVrh.10MC1 or AAVrh.10PHF-1 genomes. The full length antibody expression cassette is flanked by the two inverted terminal repeats of AAV serotype 2 (ITR) and encapsidation signal (ψ). The expression cassette comprises: the human cytomegalovirus (CMV) enhancer; the chicken β-actin promoter; splice donor and 5′ end of intron; the 3′ end of the rabbit β-globin intron and splice acceptor, the full length MC1 or PHF-1 antibody sequence expressed in a single open reading frame (ORF) with an optimized Kozak sequence; and the polyadenylation/transcription stop signal from rabbit β-globin. The full length antibody ORF includes the IgG1 leader peptide and variable and constant regions (heavy chain) in frame with the Ig_{κ} leader peptide, variable and constant regions (light chain) by inclusion of a furin cleavage recognition sequence upstream of a 2A cis-acting hydrolase element (Furin 2A).

FIG. 2. Expression of PHF-1 and MC1 in supernatant of 293T cells 48 h after transfection with pAAVPHF-1 or pAAVmC1 plasmid. A). pAAVPHF-1 transfected cells produce PHF-1 full length antibody that is secreted to the extracellular media. Western blot lanes show supernatant from pAAVPHF-1, pAAVαPCRV antibody control and Mock transfected cells. Arrows indicate antibody heavy and light chains. PHF-1 and control antibodies were detected using a goat anti-mouse IgG antibody conjugated with Horseradish Peroxidase (HRP). B). PHF-1 from the supernatant of transfected cells binds to pathogenic tau from Alzheimer's disease (AD) brain lysates. Brain lysates from healthy and AD patients were separated by SDS PAGE and assayed by Western blot using cell culture supernatants from pAAVPHF-1 transfected 293T cells as a primary antibody and a goat anti-mouse IgG antibody conjugated to HRP as secondary antibody. Arrows indicated the three expected bands for hyperphosphorylated/pathogenic Tau (P-Tau). C). MC1 from the supernatant of transfected cells binds to pathogenic tau from Alzheimer's disease (AD) brain lysates. Brain lysates from healthy and AD patients were separated by SDS PAGE and assayed by Western blot using cell culture supernatants from pAAVMC1transfected 293T cells as a primary antibody and a goat anti-mouse IgG antibody conjugated to HRP as secondary antibody. Arrows indicated the three expected bands for hyperphosphorylated/pathogenic Tau (P-Tau).

FIG. 3. Expression of PHF-1 antibody in C57BI/6 mice 6 wk after delivery of AAVrh.10PHF-1. Mice received 10¹⁰ gc of AAVrh.10PHF-1 or AAVrh.10mCherry control stereotactically into hippocampus. Six weeks after vector administration, transgene distribution was evaluated. A). mCherry (red fluorescence) distribution in hippocampus of control mice 6 weeks after administration of 10¹⁰ gc AAVrh.10mCherry. B). Expression of PHF-1 in mouse brain lysates measured by RT-PCR. The arrows point to the specific amplification band for the PHF-1 administered brain lysate and the 18S endogenous control.

FIG. 4. Expression of PHF-1 and MC1 antibodies in C57 BI/6 brain hippocampus lysates after single administration of AAVrh.10PHF-1 or AAVrh.10MC1. Mice received 10¹⁰ gc of AAVrh.10PHF-1, AAVrh.10MC1, or AAVrh.10mCherry control, stereotactically into hippocampus. Three to six weeks after vector administration, hippocampus was extracted, homogenized, and lysate was evaluated for antibody expression by ELISA. Plates were coated with paired helical filamentous tau (PHF-Tau) protein isolated from AD brains. Serial dilutions of brain lysates were added to the wells and MC1 or PHF-1 antibody binding was measured using an anti-mouse antibody conjugated to HRP and a HRP substrate. Absorbance was measured at 450 nm (OD450). A. PHF-1 antibody levels in mouse hippocampus lysate 6 weeks after administration of 10¹⁰ gc AAVrh.10PHF-1. B. MC1 antibody levels in mouse hippocampus lysate 3 weeks after administration of 10¹⁰ gc AA-Vrh.10MC1. Brain (hippocampus) lysates from animals administered 10¹⁰ gc AAVrh.10mCherry were used as control.

FIGS. 5A-5F. A). Sequence 1: PHF-1 full length Furin 2A antibody nucleotide sequence (SEQ ID NO:1). B). Sequence 2: MC1 full length Furin 2A antibody nucleotide sequence (SEQ ID NO:2). C). Sequence 3: PHF-1 full length Furin 2A antibody amino acid sequence (SEQ ID NO:3). D). Sequence 4: MC1 full length Furin 2A antibody amino acid sequence (SEQ ID NO:4). E). Sequence 5: PHF-1 full length Furin 2A antibody optimized nucleotide sequence(SEQ ID NO:5). F). Sequence 6: MC1 full length Furin 2A antibody optimized nucleotide sequence (SEQ ID NO:6).

### DETAILED DESCRIPTION

In the following description, reference is made to the accompanying drawings that form a part hereof, and in which is shown by way of illustration specific embodiments which may be practiced. These embodiments are described in detail to enable those skilled in the art to practice the invention, and it is to be understood that other embodiments may be utilized and that logical changes may be made without departing from the scope of the present invention. The following description of example embodiments is, therefore, not to be taken in a limited sense, and the scope of the present invention is defined by the appended claims.

### Definitions

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest), a coding sequence of interest in vaccine development (such as a polynucleotide expressing a protein, polypeptide or peptide suitable for eliciting an immune response in a mammal), and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by heterologousization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “infectious” virus or viral particle is one that comprises a polynucleotide component which it is capable of delivering into a cell for which the viral species is trophic. The term does not necessarily imply any replication capacity of the virus.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. Increasing enrichments of the embodiments of this invention are increasingly preferred. Thus, for example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use in the present invention generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

“Host cells,” “cell lines,” “cell cultures,” “packaging cell line” and other such terms denote higher eukaryotic cells, such as mammalian cells including human cells, useful in the present invention, e.g., to produce recombinant virus or recombinant fusion polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphonylation, lipidation, or conjugation with a labeling component.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells of the present invention are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 85%, 90%, 95% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

“Conservative” amino acid substitutions are, for example, aspartic-glutamic as polar acidic amino acids; lysine/arginine/histidine as polar basic amino acids; leucinelisoleucine/methionine/valinelalanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gin, his, lys, arg; (5) residues that influence chain orientation: gly, pro; and (6) aromatic; trp, tyr, phe.

The invention also envisions polypeptides with non-conservative substitutions. Non-conservative substitutions entail exchanging a member of one of the classes described above for another.

### Nucleic Acid Sequence Which Encodes an Antibody Directed Against Tau

The invention provides an isolated nucleic acid sequence which encodes an antibody directed against tau.

By “tau” is meant (i) the microtubule-associated protein tau (MAPT) (UniProtKB/Swiss-Prot: TAU_HUMAN, P10636), which has a size of 758 amino acids and molecular weight of 78878 Da; (ii) alternatively spliced isoforms (including but not limited to UniProtKB/Swiss-Prot reference proteins P10636-1, P10636-2, P10636-3, P10636-4, P10636-5, P10636-6, P10636-7, P10636-8, and P10636-9); (iii) homologs in non-human species; (iv) peptide fragments of (i)-(iii); (v) post-translationally modified proteins or peptides of (i)-(iv), including but not not limited to phosphorylations at serine and threonine residues, ubiquitinations, glycations, and sialylations; (vi) alternate conformations of the proteins and peptides of (i)-(v).

“Nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to, methylated and/or capped polynucleotides.

One of ordinary skill in the art will appreciate that an antibody consists of four polypeptides: two identical copies of a heavy (H) chain polypeptide and two copies of a light (L) chain polypeptide. Each of the heavy chains contains one N-terminal variable (V_{H}) region and three C-terminal constant (CH1, CH2 and CH3) regions, and each light chain contains one N-terminal variable (V_{L}) region and one C-terminal constant (C_{L}) region. The variable regions of each pair of light and heavy chains form the antigen binding site of an antibody. The nucleic acid sequence which encodes an antibody directed against tau can comprise one or more nucleic acid sequences, each of which encodes one or more of the heavy and/or light chain polypeptides of an anti-tau antibody. In this respect, the nucleic acid sequence which encodes an antibody directed against tau can comprise a single nucleic acid sequence that encodes the two heavy chain polypeptides and the two light chain polypeptides of an anti-tau antibody. Alternatively, the nucleic acid sequence which encodes an antibody directed against tau can comprise a first nucleic acid sequence that encodes both heavy chain polypeptides of an anti-tau antibody, and a second nucleic acid sequence that encodes both light chain polypeptides of an anti-tau antibody. In yet another embodiment, the nucleic acid sequence which encodes an antibody directed against tau can comprise a first nucleic acid sequence encoding a first heavy chain polypeptide of an anti-tau antibody, a second nucleic acid sequence encoding a second heavy chain polypeptide of an anti-tau antibody, a third nucleic acid sequence encoding a first light chain polypeptide of an anti-tau antibody, and a fourth nucleic acid sequence encoding a second light chain polypeptide of an anti-tau antibody.

In another embodiment, the nucleic acid sequence which encodes an antibody directed against tau encodes an antigen-binding fragment (also referred to as an “antibody fragment”) of an anti-tau antibody. The term “antigen-binding fragment” refers to one or more fragments of an antibody that retain the ability to specifically bind to an antigen (e.g., tau) (see, generally, Holliger and Hudson 2005). Examples of antigen-binding fragments include but are not limited to (i) a Fab fragment, which is a monovalent fragment consisting of the V_{L}, V_{H}, C_{L}, and C_{H1} domains; (ii) a F(ab′)2 fragment, which is a bivalent fragment comprising two Fab fragments linked by a disulfide bridge at the hinge region; and (iii) a Fv fragment consisting of the V_{L} and V_{H} domains of a single arm of an antibody. In one embodiment, the nucleic acid sequence which encodes an antibody directed against tau can comprise a nucleic acid sequence encoding a Fab fragment of an anti-tau antibody.

In one embodiment, the nucleic acid sequence can encode the tau-binding monoclonal antibody MC1 or a fragment thereof. In one embodiment, the nucleic acid sequence can encode the tau-binding monoclonal antibody PHF1 or a fragment thereof. The MC1 and PHF1 antibodies against tau have been previously shown to reduce tau pathology in P301L and P301S mice following passive immunization (Chai et al., 2012). MC1 is described in published PCT International Application WO 9620218) (incorporated in its entirety by reference), and deposited in terms of its source, secreting hybridoma ATCC No. 11736, with the American Type Culture Collection, Rockville, Md. on Oct. 26, 1994. PHF1 mAbs were described in Greenberg et al. (1992) (incorporated in its entirety by reference).

Other anti-tau mAbs useful for the invention are known in the art, such as those disclosed in U.S. Pat. No. 7,238,788, “Antibodies to phosphorylated tau, methods of making and methods of use” by Gloria Lee (included herein in its entirety by reference) and those disclosed in PCT International Application WO1995017429, entitled “Monoclonal antibodies specific to PHF-TAU, hybridomas secreting them, antigen recognition by these antibodies and their applications,” by Marc Vandermeeren, Eugeen Vanmechelen, and Andre Van De Voorde (included herein in its entirety by reference). Other monoclonal antibodies of the invention include those listed in Table 1, some of which are also listed above, as well as HT7, T46, Tau-1, Tau-5, Tau-46, E178, phosphoS396, and MAb10417.

TABLE 1

| Tau Ab | Epitope | Reference |
|---|---|---|
| PHF-1 | Lys395-Thr427; Phosphorylated Ser | Greenberg and Davies, 1990; |
| | 396 and Ser 404 | Lewis et al., 2001; Published |
| | | PCT Application |
| | | WO199620218 (Albert |
| | | Einstein College of Medicine of |
| | | Yeshiva University) |
| AT8 | Phosphorylated Ser 202 and | Mercken, 1992a; Goedert et |
| | Thr 205 | al., 1993 |
| 12EB | Phosphorylated Ser 262 and/ | Seubert et al., 1995; Litersky |
| | or Ser 356 | et al., 1996 |
| AT100 | Phosphorylated Ser 212 and Thr 214 | Mercken, 1992a; Goedert et |
| | | al., 1993 |
| DA31 | Residues 150-190 of tau | Tamayev et al., 2010; |
| | | Schlatterer et al., 2011 |
| CP13 | P-Ser202 | Boutajangout et al 2010; |
| | | Tamayev el., 2010 |
| AT180 | Tau224-238 [P231] | Mercken, 1992a; Goedert et |
| | | al., 1993, 1994; Boimel et al |
| | | 2010 |
| BT2 | wt human tau | Mercken, 1992b |
| Ab708 | Residues 160-182 for 2N4R tau | Published PCT Application |
| | isoform; containing acetylated lysines | WO2011056300 |
| | at positions 163 and 174 and 180. | |
| ALZ50 | Ala2-Tyr18; Pro 312 - Glu342 | Published PCT Application |
| | (discontinous epitope) (Glu7, Phe8 and | WO199620218 (Albert |
| | Glu9 are absolutely required for | Einstein College of Medicine of |
| | binding) | Yeshiva University) |
| TG3 | Ser210-Ser241; Arg242_Lys281; | Published PCT Application |
| | Lys395-Thr427 (discontinous epitope) | WO199620218 (Albert |
| | | Einstein College of Medicine of |
| | | Yeshiva University) |
| TG4 | Ser210-Ser241 (Weakly also to | Published PCT Application |
| | Arg242_Lys281) | WO199620218 (Albert |
| | | Einstein College of Medicine of |
| | | Yeshiva University) |
| TG5 | Thr220 - Ser235 | Published PCT Application |
| | | WO199620218 (Albert |
| | | Einstein College of Medicine of |
| | | Yeshiva University) |
| MC1 | Ala2 - Tyr18; Pro 312 - Glu342 | Jicha et al., 1997; Published |
| | (discontinous epitope); (Glu7, Phe8 | PCT Application |
| | and Glu9 are absolutely required for | WO199620218 (Albert |
| | binding); recognizes tau in a | Einstein College of Medicine of |
| | pathological conformation | Yeshiva University) |
| MC5 | Thr220_Ser235 | Published PCT Application |
| | | WO199620218 (Albert |
| | | Einstein College of Medicine of |
| | | Yeshiva University) |
| MC6 | Thr220-Ser235 | Published PCT Application |
| | | WO199620218 (Albert |
| | | Einstein College of Medicine of |
| | | Yeshiva University) |
| MC15 | Arg230_ Lys240 | Published PCT Application |
| | | WO199620218 (Albert |
| | | Einstein College of Medicine of |
| | | Yeshiva University) |
| YP3 | phosphorylation of tau at | Published PCT Application |
| | tyr394 and at ser396 | WO2007019273 (Albert |
| | | Einstein College of Medicine of |
| | | Yeshiva University) |
| YP4 | phosphorylation of tau at | Published PCT Application |
| | tyr394 and at ser396 | WO2007019273 (Albert |
| | | Einstein College of Medicine of |
| | | Yeshiva University) |
| YP21 | phosphorylation of tau | Published PCT Application |
| | at at tyr310 | WO2007019273 (Albert |
| | | Einstein College of Medicine of |
| | | Yeshiva University) |
| AT120 | wt tau | Vandermeeren M, et al 1993 |

In an embodiment, the nucleic acid sequence which encodes an antibody against tau recognized a phosphorylated epitope of tau. In an embodiment, the nucleic acid sequence which encodes an antibody against tau recognized a tau that is in a pathological conformation.

An antibody, or antigen-binding fragment thereof, can be obtained by any means, including via in vitro sources (e.g., a hybridoma or a cell line producing an antibody recombinantly) and in vivo sources (e.g., rodents). Methods for generating antibodies are known in the art and are described in, for example, Köhler and Milstein, Eur. J. Immunol., 5:511 (1976); Harlow and Lane (eds.), Antibodies: A Laboratory Manual, CSH Press (1988); and C. A. Janeway et al. (eds.), Immunobiology, 5th Ed., Garland Publishing, New York, N.Y. (2001)). In certain embodiments, a human antibody or a chimeric antibody can be generated using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the HUMAB-MOUSE™, the Kirin TC MOUSE™, and the KM-MOUSE™ (see, e.g., Lonberg, Nat. Biotechnol., 23(9):1117 (2005), and Lonberg, Handb. Exp. Pharmacal., 181:69 (2008)).

The nucleic acid sequence which encodes an antibody directed against tau, or an antigen-binding fragment thereof, can be generated using methods known in the art. For example, nucleic acid sequences, polypeptides, and proteins can be recombinantly produced using standard recombinant DNA methodology (see, e.g., Sambrook et al., Molecular Clonind: A Laboratory Manual, 3rd ed., Cold Spring Harbor Press, Cold Spring Harbor, N.Y., 2001; and Ausubel et al., Current Protocols in Molecular Biology, Greene Publishing Associates and John Wiley & Sons, NY, 1994). Further, a synthetically produced the nucleic acid sequence which encodes an antibody directed against tau, or an antigen-binding fragment thereof, can be isolated and/or purified from a source, such as a bacterium, an insect, or a mammal, e.g., a rat, a human, etc. Methods of isolation and purification are well-known in the art. Alternatively, the nucleic acid sequences described herein can be commercially synthesized. In this respect, the nucleic acid sequence can be synthetic, recombinant, isolated, and/or purified.

The nucleic acid sequence which encodes an antibody directed against tau may be identified by extracting RNA from the available antibody producing hybridoma cells. cDNA is produced by reverse transcription and PCR amplification of the light and heavy chains and is carried out using a rapid amplification of cDNA ends (RACE) strategy in combination with specific primers for conserved regions in the constant domains.

The nucleic acid sequence which encodes an antibody directed against tau may also be fully or partly humanized by means known in the art. For example, an antibody chimera may be created by substituting DNA encoding the mouse Fc region of the antibody with that of cDNA encoding for human.

The Fab portion of the molecule may also be humanized by selectively altering the DNA of non-CDR portions of the Fab sequence that differ from those in humans by exchanging the sequences for the appropriate individual amino acids.

Alternatively, humanization may be achieved by insertion of the appropriate CDR coding segments into a human antibody “scaffold”.

Resulting antibody DNA sequences may be optimized for high expression levels in mammalian cells through removal of RNA instability elements, a is known in the art.

In an embodiment, a nucleic acid sequence which encodes an antibody directed against tau, may be expressed under the control of a single promoter in a 1:1 ratio using a 2A (Chysel) self-cleavable sequence. The 2A sequence self-cleaves during protein translation and leaves a short tail of amino acids in the C-terminus of the upstream protein. A Furin cleavage recognition site may be added between the 2A sequence and the upstream gene to assure removal of the remaining amino acids. Plasmids expressing the correct inserts may be identified by DNA sequencing and by antibody specific binding using western analysis and ELISA assays.

### Gene Transfer Vectors

The invention also provides a gene transfer vector comprising a nucleic acid sequence which encodes a monoclonal antibody directed against tau. The invention further provides a method of producing an immune response against tau in a mammal, which method comprises administering to the mammal the above-described gene transfer vector. Various aspects of the inventive gene transfer vector and method are discussed below. Although each parameter is discussed separately, the inventive gene transfer vector and method comprise combinations of the parameters set forth below to evoke protection against a tau pathology. Accordingly, any combination of parameters can be used according to the inventive gene transfer vector and the inventive method.

A “gene transfer vector” is any molecule or composition that has the ability to carry a heterologous nucleic acid sequence into a suitable host cell where synthesis of the encoded protein takes place. Typically, a gene transfer vector is a nucleic acid molecule that has been engineered, using recombinant DNA techniques that are known in the art, to incorporate the heterologous nucleic acid sequence. Desirably, the gene transfer vector is comprised of DNA. Examples of suitable DNA-based gene transfer vectors include plasmids and viral vectors. However, gene transfer vectors that are not based on nucleic acids, such as liposomes, are also known and used in the art. The inventive gene transfer vector can be based on a single type of nucleic acid (e.g., a plasmid) or non-nucleic acid molecule (e.g., a lipid or a polymer). The inventive gene transfer vector can be integrated into the host cell genome, or can be present in the host cell in the form of an episome.

In one embodiment, the gene transfer vector is a viral vector. Suitable viral vectors include, for example, retroviral vectors, herpes simplex virus (HSV)-based vectors, parvovirus-based vectors, e.g., adeno-associated virus (AAV)-based vectors, AAV-adenoviral chimeric vectors, and adenovirus-based vectors. These viral vectors can be prepared using standard recombinant DNA techniques described in, for example, Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994).

In an embodiment, the invention provides an adeno-associated virus (AAV) vector which comprises, consists essentially of, or consists of a nucleic acid sequence encoding an antibody that binds to tau, or an antigen-binding fragment thereof. When the inventive AAV vector consists essentially of a nucleic acid sequence encoding an antibody that binds to tau, additional components can be included that do not materially affect the AAV vector (e.g., genetic elements such as poly(A) sequences or restriction enzyme sites that facilitate manipulation of the vector in vitro). When the AAV vector consists of a nucleic acid sequence which encodes a monoclonal antibody directed against tau, the AAV vector does not comprise any additional components (i.e., components that are not endogenous to AAV and are not required to effect expression of the nucleic acid sequence to thereby provide the antibody).

Adeno-associated virus is a member of the Parvoviridae family and comprises a linear, single-stranded DNA genome of less than about 5,000 nucleotides. AAV requires co-infection with a helper virus (i.e., an adenovirus or a herpes virus), or expression of helper genes, for efficient replication. AAV vectors used for administration of therapeutic nucleic acids typically have approximately 96% of the parental genome deleted, such that only the terminal repeats (ITRs), which contain recognition signals for DNA replication and packaging, remain. This eliminates immunologic or toxic side effects due to expression of viral genes. In addition, delivering specific AAV proteins to producing cells enables integration of the AAV vector comprising AAV ITRs into a specific region of the cellular genome, if desired (see, e.g., U.S. Pat. Nos. 6,342,390 and 6,821,511). Host cells comprising an integrated AAV genome show no change in cell growth or morphology (see, for example, U.S. Pat. No. 4,797,368).

The AAV ITRs flank the unique coding nucleotide sequences for the non-structural replication (Rep) proteins and the structural capsid (Cap) proteins (also known as virion proteins (VPs)). The terminal 145 nucleotides are self-complementary and are organized so that an energetically stable intramolecular duplex forming a T-shaped hairpin may be formed. These hairpin structures function as an origin for viral DNA replication by serving as primers for the cellular DNA polymerase complex. The Rep genes encode the Rep proteins Rep78, Rep68, Rep52, and Rep40. Rep78 and Rep68 are transcribed from the p5 promoter, and Rep 52 and Rep40 are transcribed from the p19 promoter. The Rep78 and Rep68 proteins are multifunctional DNA binding proteins that perform helicase and nickase functions during productive replication to allow for the resolution of AAV termini (see, e.g., Im et al., Cell, 61:447 (1990)). These proteins also regulate transcription from endogenous AAV promoters and promoters within helper viruses (see, e.g., Pereira et al., J. Virol., 71:1079 (1997)). The other Rep proteins modify the function of Rep78 and Rep68. The cap genes encode the capsid proteins VP1, VP2, and VP3. The cap genes are transcribed from the p40 promoter.

The AAV vector may be generated using any AAV serotype known in the art. Several AAV serotypes and over 100 AAV variants have been isolated from adenovirus stocks or from human or nonhuman primate tissues (reviewed in, e.g., Wu et al., Molecular Therapy, 14(3): 316 (2006)). Generally, the AAV serotypes have genomic sequences of significant homology at the nucleic acid sequence and amino acid sequence levels, such that different serotypes have an identical set of genetic functions, produce virions which are essentially physically and functionally equivalent, and replicate and assemble by practically identical mechanisms. AAV serotypes 1-6 and 7-9 are defined as “true” serotypes, in that they do not efficiently cross-react with neutralizing sera specific for all other existing and characterized serotypes. In contrast, AAV serotypes 6, 10 (also referred to as Rh10), and 11 are considered “variant” serotypes as they do not adhere to the definition of a “true” serotype. AAV serotype 2 (AAV2) has been used extensively for gene therapy applications due to its lack of pathogenicity, wide range of infectivity, and ability to establish long-term transgene expression (see, e.g., Carter, Hum. Gene Ther., 16:541 (2005); and Wu et al., supra). Genome sequences of various AAV serotypes and comparisons thereof are disclosed in, for example, GenBank Accession numbers U89790, J01901, AF043303, and AF085716; Chiorini et al., J. Virol., 71:6823 (1997); Srivastava et al., J. Virol., 45:555 (1983); Chiorini et al., J. Virol., 73:1309 (1999); Rutledge et al., J. Virol., 72:309 (1998); and Wu et al., J. Virol. 74:8635 (2000)).

AAV rep and ITR sequences are particularly conserved across most AAV serotypes. For example, the Rep78 proteins of AAV2, AAV3A, AAV3B, AAV4, and AAV6 are reportedly about 89-93% identical (see Bantel-Schaal et al., J. Virol., 73(2):939 (1999)). It has been reported that AAV serotypes 2, 3A, 3B, and 6 share about 82% total nucleotide sequence identity at the genome level (Bantel-Schaal et al., supra). Moreover, the rep sequences and ITRs of many AAV serotypes are known to efficiently cross-complement (e.g., functionally substitute) corresponding sequences from other serotypes during production of AAV particles in mammalian cells.

Generally, the cap proteins, which determine the cellular tropicity of the AAV particle, and related cap protein-encoding sequences, are significantly less conserved than Rep genes across different AAV serotypes. In view of the ability Rep and ITR sequences to cross-complement corresponding sequences of other serotypes, the AAV vector can comprise a mixture of serotypes and thereby be a “chimeric” or “pseudotyped” AAV vector. A chimeric AAV vector typically comprises AAV capsid proteins derived from two or more (e.g., 2, 3, 4, etc.) different AAV serotypes. In contrast, a pseudotyped AAV vector comprises one or more ITRs of one AAV serotype packaged into a capsid of another AAV serotype. Chimeric and pseudotyped AAV vectors are further described in, for example, U.S. Pat. No. 6,723,551; Flotte, Mol. Ther. 13(1):1 (2006); Gao et al., J. Virol. 78:6381 (2004); Gao et al., Proc. Natl. Acad. Sci. USA, 99:11854 (2002); De et al., Mol. Ther., 13:67 (2006); and Gao et al., Mol. Ther., 13:77 (2006).

In one embodiment, the AAV vector is generated using an AAV that infects humans (e.g., AAV2). Alternatively, the AAV vector is generated using an AAV that infects non-human primates, such as, for example, the great apes (e.g., chimpanzees), Old World monkeys (e.g., macaques), and New World monkeys (e.g., marmosets). In one embodiment, the AAV vector is generated using an AAV that infects a non-human primate pseudotyped with an AAV that infects humans. Examples of such pseudotyped AAV vectors are disclosed in, e.g., Cearley et al., Molecular Therapy, 13:528 (2006). In one embodiment, an AAV vector can be generated which comprises a capsid protein from an AAV that infects rhesus macaques pseudotyped with AAV2 inverted terminal repeats (ITRs). In a particular embodiment, the inventive AAV vector comprises a capsid protein from AAV10 (also referred to as “AAVrh.10”), which infects rhesus macaques pseudotyped with AAV2 ITRs (see, e.g., Watanabe et al., Gene Ther., 17(8):1042 (2010); and Mao et al., Hum. Gene Therapy, 22:1525 (2011)).

In addition to the nucleic acid sequence encoding an antibody against tau, or an antigen-binding fragment thereof, the AAV vector may comprise expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and described in, for example, Goeddel, Gene Expression Technology: Methods in Enzymology, Vol. 185, Academic Press, San Diego, Calif. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system (U.S. Pat. Nos. 5,464,758 and 5,814,618), the Ecdysone inducible system (No et al., Proc. Natl. Acad. Sci., 93:3346 (1996)), the T-REXTM system (Invitrogen, Carlsbad, Calif.), LACSWITCH™ System (Stratagene, San Diego, Calif.), and the Cre-ERT tamoxifen inducible recombinase system (Indra et al., Nuc. Acid. Res. 27:4324 (1999); Nuc. Acid. Res., 28:e99 (2000); U.S. Pat. No. 7,112,715; and Kramer & Fussenegger, Methods Mol. Biol., 308:123 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. In one embodiment, the nucleic acid sequence encoding an antibody against tau, or an antigen-binding fragment thereof, is operably linked to a CMV enhancer/chicken beta-actin promoter (also referred to as a “CAG promoter”) (see, e.g., Niwa et al., Gene, 108:193 (1991); Daly et al., Proc. Natl. Acad. Sci. U.S.A., 96:2296 (1999); and Sondhi et al., Mol. Ther., 15:481 (2007)).

Typically AAV vectors are produced using well characterized plasmids. For example, human embryonic kidney 293T cells are transfected with one of the transgene specific plasmids and another plasmid containing the adenovirus helper and AAV rep and cap genes (specific to AAVrh.10, 8 or 9 as required). After 72 hours, the cells are harvested and the vector is released from the cells by five freeze/thaw cycles. Subsequent centrifugation and benzonase treatment removes cellular debris and unencapsidated DNA. Iodixanol gradients and ion exchange columns may be used to further purify each AAV vector. Next, the purified vector is concentrated by a size exclusion centrifuge spin column to the required concentration. Finally, the buffer is exchanged to create the final vector products formulated (for example) in 1× phosphate buffered saline. The viral titers may be measured by TaqMan® real-time PCR and the viral purity may be assessed by SDS-PAGE.

### Pharmaceutical Compositions and Delivery

The invention provides a composition comprising, consisting essentially of, or consisting of the above-described gene transfer vector and a pharmaceutically acceptable (e.g., physiologically acceptable) carrier. When the composition consists essentially of the inventive gene transfer vector and a pharmaceutically acceptable carrier, additional components can be included that do not materially affect the composition (e.g., adjuvants, buffers, stabilizers, anti-inflammatory agents, solubilizers, preservatives, etc.). When the composition consists of the inventive gene transfer vector and the pharmaceutically acceptable carrier, the composition does not comprise any additional components. Any suitable carrier can be used within the context of the invention, and such carriers are well known in the art. The choice of carrier will be determined, in part, by the particular site to which the composition may be administered and the particular method used to administer the composition. The composition optionally can be sterile with the exception of the gene transfer vector described herein. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, Pa. (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the inventive gene transfer vector is administered in a composition formulated to protect the gene transfer vector from damage prior to administration. For example, the composition can be formulated to reduce loss of the gene transfer vector on devices used to prepare, store, or administer the gene transfer vector, such as glassware, syringes, or needles. The composition can be formulated to decrease the light sensitivity and/or temperature sensitivity of the gene transfer vector. To this end, the composition may comprise a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the gene transfer vector, facilitate administration, and increase the efficiency of the inventive method. Formulations for gene transfer vector-containing compositions are further described in, for example, Wright et al., Curr. Opin. Drug Discov. Devel., 6(2): 174-178 (2003) and Wright et al., Molecular Therapy, 12: 171-178 (2005))

The composition also can be formulated to enhance transduction efficiency. In addition, one of ordinary skill in the art will appreciate that the inventive gene transfer vector can be present in a composition with other therapeutic or biologically-active agents. For example, factors that control inflammation, such as ibuprofen or steroids, can be part of the composition to reduce swelling and inflammation associated with in vivo administration of the gene transfer vector. Immune system stimulators or adjuvants, e.g., interleukins, lipopolysaccharide, and double-stranded RNA, can be administered to enhance or modify the anti-tau immune response. Antibiotics, i.e., microbicides and fungicides, can be present to treat existing infection and/or reduce the risk of future infection, such as infection associated with gene transfer procedures.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In certain embodiments, a formulation of the present invention comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the inventive gene transfer vector. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

Delivery of the compositions comprising the inventive gene transfer vectors may be intracerebral (including but not limited to intraparenchymal, intraventricular, or intracisternal), intrathecal (including but not limited to lumbar or cisterna magna), or systemic, including but not limited to intravenous, or any combination thereof, using devices known in the art. Delivery may also be via surgical implantation of an implanted device. Intracisternal delivery of AAV.rh10-tau antibody yields a relatively non-invasive route of administration and one amenable to use in pre-symptomatic or symptomatic patients with AD or other diseases and conditions characterized by pathological tau activity.

The dose of the gene transfer vector in the composition administered to the mammal will depend on a number of factors, including the size (mass) of the mammal, the extent of any side-effects, the particular route of administration, and the like. In one embodiment, the inventive method comprises administering a “therapeutically effective amount” of the composition comprising the inventive gene transfer vector described herein. A “therapeutically effective amount” refers to an amount effective, at dosages and for periods of time necessary, to achieve a desired therapeutic result. The therapeutically effective amount may vary according to factors such as the extent of tau pathology, age, sex, and weight of the individual, and the ability of the gene transfer vector to elicit a desired response in the individual. The dose of gene transfer vector in the composition required to achieve a particular therapeutic effect typically is administered in units of vector genome copies per cell (gc/cell) or vector genome copies/per kilogram of body weight (gc/kg). One of ordinary skill in the art can readily determine an appropriate gene transfer vector dose range to treat a patient having a particular disease or disorder, based on these and other factors that are well known in the art. The therapeutically effective amount may be between 1×10¹⁰ genome copies to 1×10¹³ genome copies.

In one embodiment of the invention, the composition is administered once to the mammal. It is believed that a single administration of the composition will result in persistent expression of the anti-tau antibody in the mammal with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

The present invention provides pharmaceutically acceptable compositions which comprise a therapeutically-effective amount of gene transfer vector comprising a nucleic acid sequence which encodes an antibody directed against tau as described above.

### Neurodegenerative Diseases and Conditions.

The invention is useful to treat a subject with a medical condition or disorder that involves pathological activity of tau or changes in tau activity and/or the formation of neurofibrillary tangles (NFTs), including neurodegenerative disorders, and ischemic and traumatic brain injury. Such medical conditions and disorders include but are not limited to preclinical and clinical Alzheimer's disease (AD), mild cognitive impairment, frontotemporal dementia, traumatic brain injury (TBI), stroke, and transient ischemic attack.

Other conditions include: vascular dementia, Creutzfeldt-Jakob disease, multiple sclerosis, prion disease, Pick's disease, corticobasal degeneration, Parkinson's disease, Lewy body dementia, Progressive supranuclear palsy; Dementia pugilistica (chronic traumatic encephalopathy); frontotemporal dementia and parkinsonism linked to chromosome 17; Lytico-Bodig disease; Tangle-predominant dementia; Ganglioglioma and gangliocytoma; Meningioangiomatosis; Subacute sclerosing panencephalitis; lead encephalopathy, tuberous sclerosis, Hallervorden-Spatz disease, and lipofuscinosis; Argyrophilic grain disease; and Frontotemporal lobar degeneration.

### Subjects

The subject may be any animal, including a human and non-human animal. Non-human animals includes all vertebrates, e.g., mammals and non-mammals, such as non-human primates, sheep, dogs, cats, cows, horses, chickens, amphibians, and reptiles, although mammals are envisioned as subjects, such as non-human primates, sheep, dogs, cats, cows and horses. The subject may also be livestock such as, cattle, swine, sheep, poultry, and horses, or pets, such as dogs and cats.

Preferred subjects include human subjects suffering from or at risk for the medical diseases and conditions described herein. The subject is generally diagnosed with the condition of the subject invention by skilled artisans, such as a medical practitioner.

The methods of the invention described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods of the invention may be more appropriate for some ethnic populations such as Caucasians, especially northern European populations, as well as Asian populations.

The term subject also includes subjects of any genotype or phenotype as long as they are in need of the invention, as described above. In addition, the subject can have the genotype or phenotype for any hair color, eye color, skin color or any combination thereof. The term subject includes a subject of any body height, body weight, or any organ or body part size or shape.

The invention will be described by the following non-limiting examples.

### EXAMPLES

### Example

### In Vitro Studies

The progressive staging of tau pathology results from spreading of pathologic (misfolded) tau within various neuronal networks. The exemplary therapy disclosed herein is a biological drug directed specifically at the tau pathogenic process using an adeno-associated virus, e.g., serotype rh.10 (AAVrh.10), gene transfer to achieve sustained anti-tau monoclonal antibody expression over a wide area of the brain. MC1 and PHF-1 monoclonal antibodies were used for the construction of the AAVrh.10PHF-1 and AAVrh.10MC1 vectors.

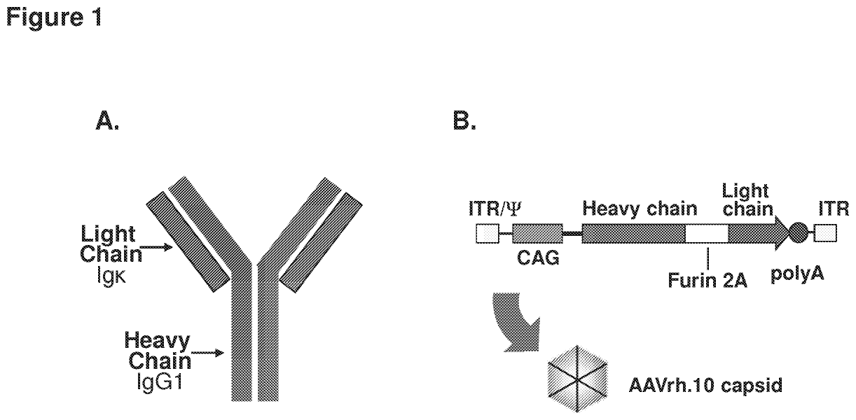

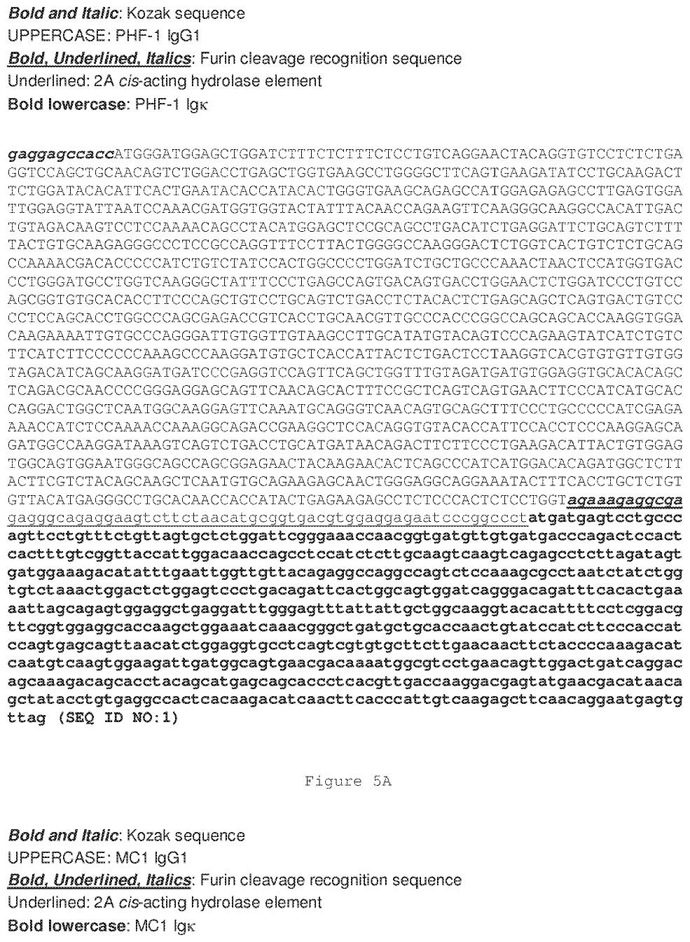

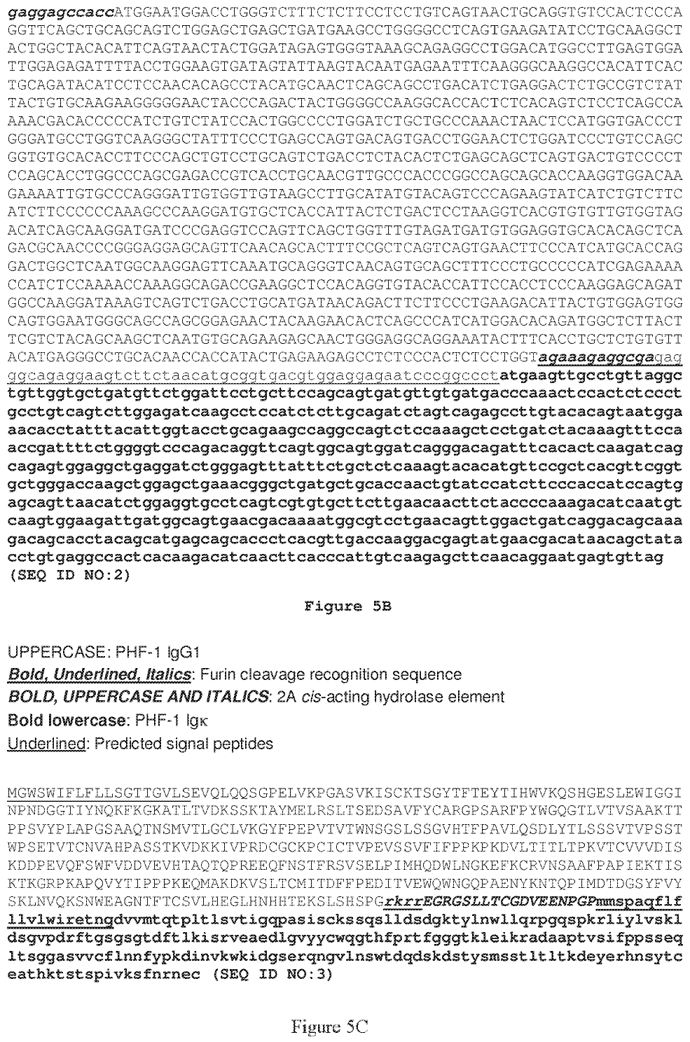

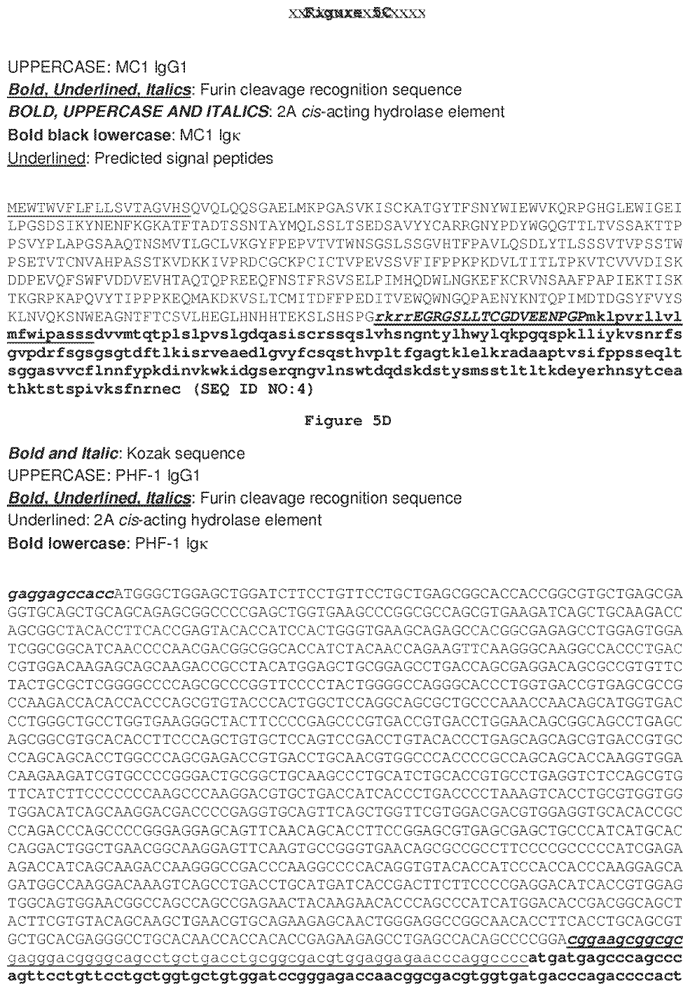

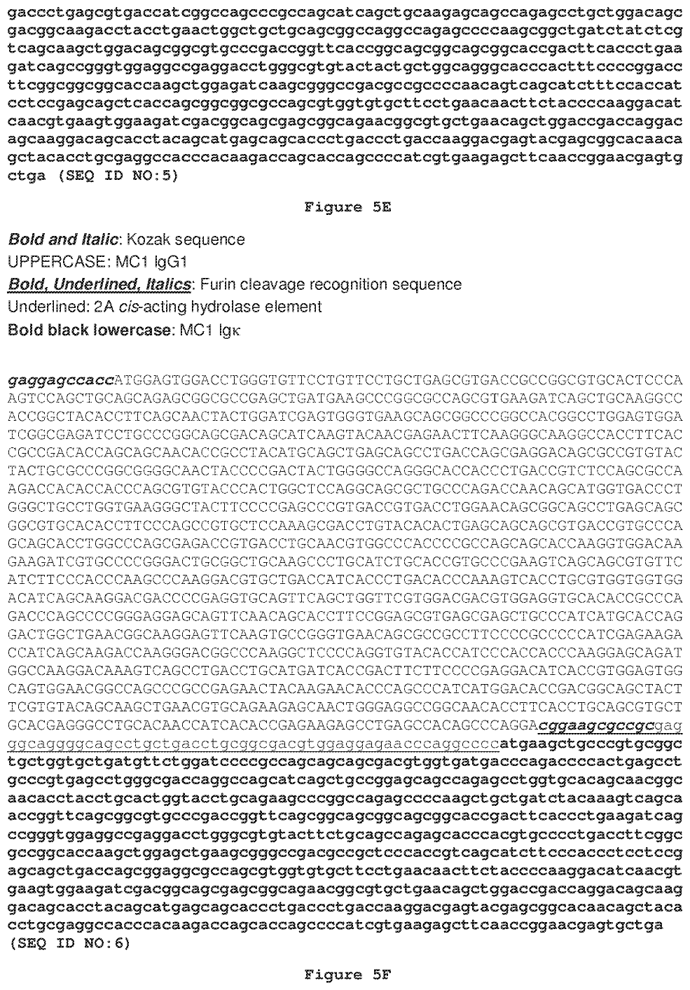

AAVrh.10MC1 and AAVrh.10PHF-1 cloning. MC1 and PHF-1 cDNA sequences were amplified from hybridoma cells (generous gift of Dr. Peter Davies) using a rapid amplification of cDNA ends (RACE) method and cloned into a pAAV plasmid. Total RNA was extracted from the PHF-1 and MC1 hybridoma cell lysates and cDNA was synthetized in two independent reactions by using primers annealing to conserved regions of the constant chains or by using random hexamers. Light and heavy chain sequences were then amplified from the cDNA using nested primers, cloned into a TOPO vector and fully sequenced. Subsequently, the full antibody constructs were assembled by overlapping PCR. Antibody light and heavy chains are expressed in a 1:1 ratio under the CAG promoter by use of a 2A cis-acting hydrolase element downstream of a furin cleavage recognition site, Furin 2A (see FIG. 1 and FIG. 5). These pAAV constructs were used to test expression in vitro and in vivo in a mouse model. Nucleotide sequences were further optimized for expression in mammalian cells by use replacement of at least some codons with those preferred (usage) in mammalian cells, removal of potential splicing signals, mRNA instability elements and high GC content regions (Sequences 5, 6 in FIG. 5).

### Results

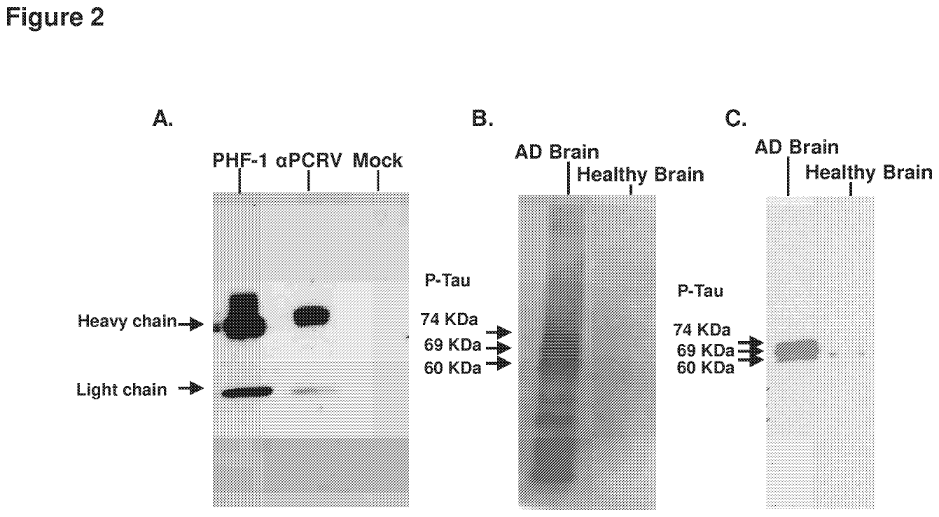

HEK 293T cells were transfected with the pAAV plasmid expressing either MC1 (pAAVMC1) or PHF-1 (pAAVPHF-1) and cell culture supernatants were assayed for presence of functional anti-Tau antibody by Western blot (FIG. 2). pAAVMC1 and pAAVrh.10PHF-1 transfected cells expressed the full length antibody and can recognize pathological tau from Alzheimer's disease brain lysates by Western assay.

### Example

### Mouse Study

AAV.rh10 is used to deliver the MC1 anti-tau antibodies directly to the CNS, thus bypassing the blood: brain barrier (BBB). As described above, cDNA encoding the light and heavy chains of MC1 antibody or PHF1 antibody was isolated from the hybridomas producing these antibodies, and construct an AAV.rh10 viral vector that contains nucleic acid encoding light and heavy chains of the antibody. AAV.rh10 MC1 or PHF1 virus was produced in HEK 293 cells.

Instead of administering the antibody directly, AAVrh.10 MC1 virus and AAV.rh.10 PHF1 virus are administered to each of 15 P301S mice via the intraventricular route at 10¹¹ particles at 2 months of age because at the cellular level, pathological tau can be observed in many brain areas including the cerebral cortex, hippocampus and brainstem at 5-6 months of age in P301S mice. A group of 15 P301S mice is administered AAV.rh10 GFP as controls. Four months after treatment, motor behavior is evaluated using the rotorod with each of the antibody-treated and the non-treated mice. Mice are then sacrificed and the brain tissue is harvested. Half of the brain is used for biochemical analysis and the other half for immunohistochemical analysis (IHC). The effect of the anti-tau antibody AAV construct is evaluated by examining and comparing tau pathology between antibody-treated and non-treated mice using biochemical (AT8- or AT100 ELISAs and western blots) and IHC analyses.

Intracisternal and combination intravenous/intracisternal delivery of the AAV anti-tau antibody, e.g., following administration into the subarachnoid space, is also evaluated. This route of delivery is less invasive when compared to that of direct intracerebral injection to the brain or even intraventricular administration. For example, AAV.rh,10 MC1 virus and AAV.rh.10 PHF1 virus is administered to each of 15 P301S mice via the intracisternal and the combination intravenous/intracisternal route at 10¹¹ particles per mouse. A group of 15 P301S mice is administered AAV.rh10 GFP as controls for each of the delivery arms. Four months after treatment, motor behavior is evaluated using the rotorod with each of the antibody-treated and the non-treated mice. Mice are then sacrificed and the brain tissue is harvested. Half of the brain is used for biochemical analysis and the other half for immunohistochemical analysis (IHC). The effect of the anti-tau antibody AAV construct is evaluated by examining and comparing tau pathology between antibody-treated and non-treated mice using biochemical (AT8- or AT100 ELISAs and western blots) and IHC analyses.

The treated mice perform significantly better than controls in the rotarod test, and there is a highly significant reduction in the amount of tau with respect to controls.

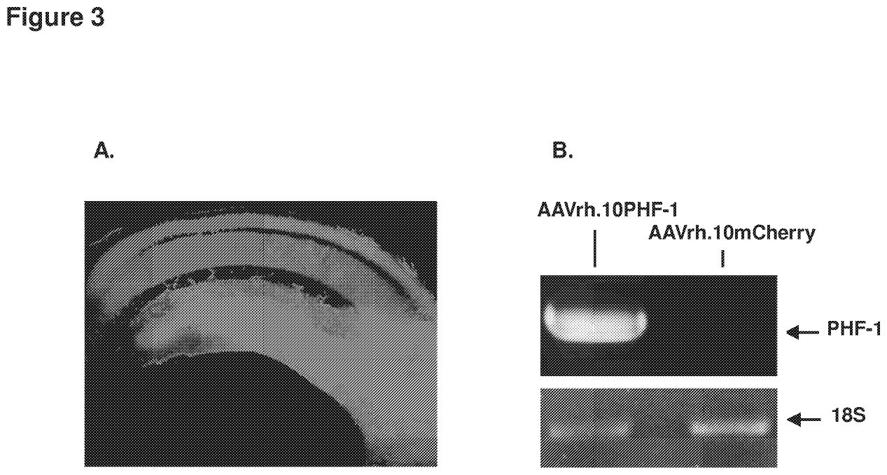

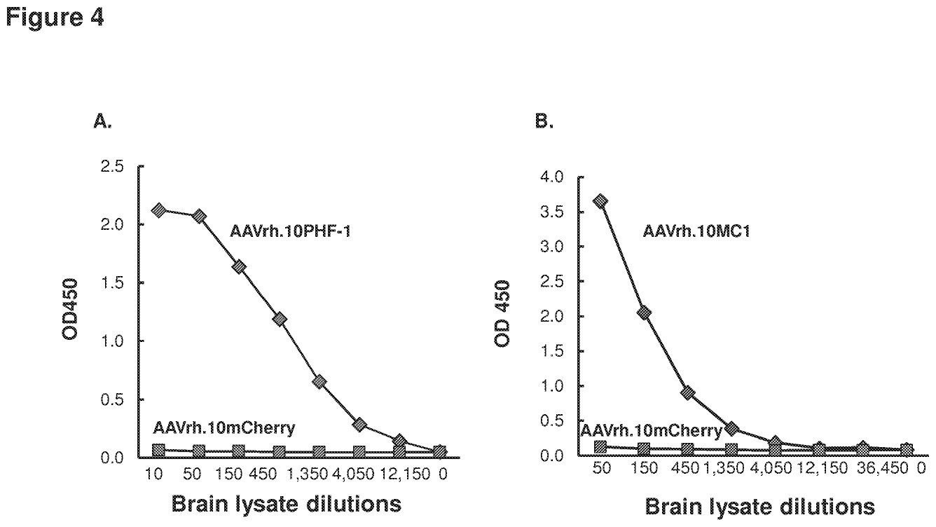

In another embodiment, MC1 and PHF-1 antibody expression was evaluated in vivo after delivery of the AAVrh.10 anti-Tau vectors into the mouse hippocampus. The PHF-1 and MC1 expression cassettes were packaged into the AAVrh.10 capsid and purified by chromatography techniques. After purification, 10¹⁰ genome copies (gc) of either AAVrh.10MC1 or AAVrh.10PHF-1 vector were injected into the hippocampus of C57BI/6 mice. As control, a group of mice received 10¹⁰ of an AAVrh.10 vector expressing the mCherry reporter gene. Vectors were delivered into the hippocampus and transgene expression was evaluated in brain lysates by RT-PCR and ELISA. The AAVrh.10 vectors were broadly distributed through the hippocampus of injected mice (FIG. 3A). PHF-1 expression was confirmed in brain lysates by RT-PCR (FIG. 3B) and high antibody titers in the brain lysates were confirmed by ELISA 6 weeks after administration of AAVrh.10PHF-1 (FIG. 4A) and 3 weeks after administration of AAVrh.10MC1 (FIG. 4B). Thus, AAVrh.10MC1 and AAVrh.10PHF-1 express functional full length antibody in vivo after delivery into the mouse hippocampus.

The treated mice perform significantly better than controls in the rotarod test, and there is a highly significant reduction in the amount of tau with respect to controls.

### REFERENCES

Asuni, A. A., Boutajangout, A., Quartermain, D., Sigurdsson, E. M. Immunotherapy targeting pathological tau conformers in a tangle mouse model reduces brain pathology with associated functional improvements. J. Neurosci. 27, 9115-9129, (2007).
Boutajangout A et al. Passive immunization targeting pathological phospho-tau protein in a mouse model reduces functional decline and clears tau aggregates from the brain. J Neurochem 2010; 118, 658-667
Boutajangout, A., Ingadottir, J., Davies, P. and Sigurdsson, E. M. Passive immunization targeting pathological phospho-tau protein in a mouse model reduces functional decline and clears tau aggregates from the brain. J. Neurochem. 118, 658-667, (2011).
Boutajangout, A., Quartermain, D., and Sigurdsson, E. M. Immunotherapy targeting pathological tau prevents cognitive decline in a new tangle mouse model. J. Neurosci. 30, 16559-16566, (2010).
Braak, H. and Braak, E. Neuropathological staging of Alzheimer-related changes. Acta. Neuropathol. 82: 239-259, (1991).
Calignon A et al. Propagation of tau pathology in a model of early Alzheimer's disease. Neuron. 2012; 73: 685-697
Chai X et al. Passive immunization with anti-tau antibodies in two transgenic models. J Bio Chem 2012; 286: 34457-34467
Chai, X., Wu, S., Murray, T. K., Kinley, R., Cella, C. V., Sims, H., Buckner, N., Hanmer, J., Davies, P., O'Neill, M. J J., Hutton, M. L and Citron, M. Passive immunization with anti-tau antibodies in two transgenic models. J. Bio. Chem. 286, 34457-34467, (2012).
Clavaguera F et al. Transmission and spreading of tauopathy in transgenic mouse brain. Nat Cell Biol 2009; 11:909-14.
Clavaguera, F., Bolmont, T., Crowther, R. A., Abramowski, D., Frank, S., Probst, A., Fraser, G., Stalder, A. K., Beibel, M., Staufenbiel, M., Jucker, M., Goedert, M., Tolnay, M. Transmission and spreading of tauopathy in transgenic mouse brain. Nat. Cell. Biol. 11: 909-913, (2009).
Frost, B., Jacks, R. L., Diamond, M. I. Propagation of tau misfolding from the outside to the inside of a cell. J. Biol. Chem. 284, 12845-12852 (2009).
Greenberg S G, Davies P, Schein J D, Binder L I. Hydrofluoric acid-treated tau PHF proteins display the same biochemical properties as normal tau. J Biol Chem. 267(1):564-9. (1992)
Holliger P, Hudson P J. Engineered antibody fragments and the rise of single domains. Nat Biotechnol. 2005 September; 23(9):1126-36.
Kfoury, N., Holmes, B. B., Jiang, H., Holtzman, D. M. and Diamond, M. I. Trans-cellular propagation of tau aggregation by fibrillar species. J. Biol. Chem. 287, 19440-19451, (2012).
Liu L et al. Trans-synaptic spread of tau pathology in vivo. PLoS One 2012: 7:e31302
PCT International Application WO1995017429, entitled “Monoclonal antibodies specific PHF-TAU, hybridomas secreting them, antigen recognition by these antibodies and their applications” invented by Marc Vandermeeren, Eugeen Vanmechelen, and Andre Van De Voorde
PCT International Application WO9620218, “Antigen, Antibodies And Diagnostic Assay For Alzheimer's Disease”. Inventors: Ghanbari Hossein A, Davies Peter, and Wolozin Benjamin.
U.S. Pat. No. 7,238,788, “Antibodies to phosphorylated tau, methods of making and methods of use” and invented by Gloria Lee
Wang, G., Qiu, J., Wang, R., Krause, A., Boyer, J. L., Hackett, N. R. and Crystal, R G. Persistent expression of biologically active anti-HER2 antibody by AAVrh.10-mediated gene transfer. Cancer Gene Ther. 17, 559-570, (2010).
Watanabe, M., Boyer, J. L. and Crystal, R. G. AAVrh.10-mediated genetic delivery of bevacizumab to the pleura to provide local anti-VEGF to suppress growth of metastatic lung tumors. Gene Ther. 17, 1042-1051, (2010).
Worgall S, Sondhi D, Hackett N R, Kosofsky B, Kekatpure M V, et al. Treatment of late infantile neuronal ceroid lipofuscinosis by CNS administration of a serotype 2 adeno-associated virus expressing CLN2 cDNA. Human Gene Ther. 19, 463-474, (2008).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1-28. (canceled)

29. A method of treating Alzheimer's disease in a mammal, comprising administering to the mammal an effective amount of a composition comprising a recombinant adeno-associated virus rh10 (rAAVrh 10) comprising a heterologous promoter operably linked to a nucleic acid sequence comprising an open reading frame which encodes heavy and light Ig chains for a monoclonal antibody that, when bound to tau, binds phosphorylated serine at position 396 and phosphorylated serine at position 404 in tau.

30. The method of claim 29 wherein the composition is administered intracranially.

31. The method of claim 29 wherein the composition is administered intraventricularly.

32. The method of claim 29 wherein the composition is administered intracisternally.

33. The method of claim 29 wherein the composition is administered intravenously.

34. The method of claim 29 wherein the mammal is a human.

35. The method of claim 29 wherein the heavy chain is an IgG heavy chain or wherein the light chain is an Ig_{K} light chain.

36. The method of claim 29 wherein the open reading frame encodes a protease cleavage site between the open reading frame for the heavy chain and the open reading frame for the light chain.

37. The method of claim 29 wherein the open reading frame is operably linked to a promoter that is expressed in neurons, oligodendrocytes, glial cells or astrocytes.

38. The method of claim 29 wherein the open reading frame is operably linked to a cytomegalovirus/chicken beta-actin hybrid promoter or a glial fibrillary acidic protein promoter.


SEQUENCE LISTING (6 sequences)

SEQ ID NO: 1 - DNA, 2174 bp, Artificial Sequence
  A synthetic oligonucleotide sequence
gaggagccaccatgggatggagctggatctttctctttctcctgtcaggaactacaggtgtcctctctgaggtccagctgcaacagtctggacctgagctggtgaagcctggggcttcagtgaagatatcctgcaagacttctggatacacattcactgaatacaccatacactgggtgaagcagagccatggagagagccttgagtggattggaggtattaatccaaacgatggtggtactatttacaaccagaagttcaagggcaaggccacattgactgtagacaagtcctccaaaacagcctacatggagctccgcagcctgacatctgaggattctgcagtcttttactgtgcaagagggccctccgccaggtttccttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtagaaagaggcgagagggcagaggaagtcttctaacatgcggtgacgtggaggagaatcccggccctatgatgagtcctgcccagttcctgtttctgttagtgctctggattcgggaaaccaacggtgatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacagattcactggcagtggatcagggacagatttcacactgaaaattagcagagtggaggctgaggatttgggagtttattattgctggcaaggtacacattttcctcggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 2 - DNA, 2168 bp, Artificial Sequence
  A synthetic oligonucleotide sequence
gaggagccaccatggaatggacctgggtctttctcttcctcctgtcagtaactgcaggtgtccactcccaggttcagctgcagcagtctggagctgagctgatgaagcctggggcctcagtgaagatatcctgcaaggctactggctacacattcagtaactactggatagagtgggtaaagcagaggcctggacatggccttgagtggattggagagattttacctggaagtgatagtattaagtacaatgagaatttcaagggcaaggccacattcactgcagatacatcctccaacacagcctacatgcaactcagcagcctgacatctgaggactctgccgtctattactgtgcaagaagggggaactacccagactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtagaaagaggcgagagggcagaggaagtcttctaacatgcggtgacgtggaggagaatcccggccctatgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 3 - protein, 720 aa, Artificial Sequence
  A synthetic peptide sequence
MGWSWIFLFLLSGTTGVLSEVQLQQSGPELVKPGASVKISCKTSGYTFTEYTIHWVKQSHGESLEWIGGINPNDGGTIYNQKFKGKATLTVDKSSKTAYMELRSLTSEDSAVFYCARGPSARFPYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGRKRREGRGSLLTCGDVEENPGPMMSPAQFLFLLVLWIRETNGDVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 4 - protein, 718 aa, Artificial Sequence
  A synthetic peptide sequence
MEWTWVFLFLLSVTAGVHSQVQLQQSGAELMKPGASVKISCKATGYTFSNYWIEWVKQRPGHGLEWIGEILPGSDSIKYNENFKGKATFTADTSSNTAYMQLSSLTSEDSAVYYCARRGNYPDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGRKRREGRGSLLTCGDVEENPGPMKLPVRLLVLMFWIPASSSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - DNA, 2174 bp, Artificial Sequence
  A synthetic oligonucleotide sequence
gaggagccaccatgggctggagctggatcttcctgttcctgctgagcggcaccaccggcgtgctgagcgaggtgcagctgcagcagagcggccccgagctggtgaagcccggcgccagcgtgaagatcagctgcaagaccagcggctacaccttcaccgagtacaccatccactgggtgaagcagagccacggcgagagcctggagtggatcggcggcatcaaccccaacgacggcggcaccatctacaaccagaagttcaagggcaaggccaccctgaccgtggacaagagcagcaagaccgcctacatggagctgcggagcctgaccagcgaggacagcgccgtgttctactgcgctcggggccccagcgcccggttcccctactggggccagggcaccctggtgaccgtgagcgccgccaagaccacaccacccagcgtgtacccactggctccaggcagcgctgcccaaaccaacagcatggtgaccctgggctgcctggtgaagggctacttccccgagcccgtgaccgtgacctggaacagcggcagcctgagcagcggcgtgcacaccttcccagctgtgctccagtccgacctgtacaccctgagcagcagcgtgaccgtgcccagcagcacctggcccagcgagaccgtgacctgcaacgtggcccaccccgccagcagcaccaaggtggacaagaagatcgtgccccgggactgcggctgcaagccctgcatctgcaccgtgcctgaggtctccagcgtgttcatcttcccccccaagcccaaggacgtgctgaccatcaccctgacccctaaagtcacctgcgtggtggtggacatcagcaaggacgaccccgaggtgcagttcagctggttcgtggacgacgtggaggtgcacaccgcccagacccagccccgggaggagcagttcaacagcaccttccggagcgtgagcgagctgcccatcatgcaccaggactggctgaacggcaaggagttcaagtgccgggtgaacagcgccgccttccccgcccccatcgagaagaccatcagcaagaccaagggccgacccaaggccccacaggtgtacaccatcccaccacccaaggagcagatggccaaggacaaagtcagcctgacctgcatgatcaccgacttcttccccgaggacatcaccgtggagtggcagtggaacggccagccagccgagaactacaagaacacccagcccatcatggacaccgacggcagctacttcgtgtacagcaagctgaacgtgcagaagagcaactgggaggccggcaacaccttcacctgcagcgtgctgcacgagggcctgcacaaccaccacaccgagaagagcctgagccacagccccggacggaagcggcgcgagggacggggcagcctgctgacctgcggcgacgtggaggagaacccaggccccatgatgagcccagcccagttcctgttcctgctggtgctgtggatccgggagaccaacggcgacgtggtgatgacccagaccccactgaccctgagcgtgaccatcggccagcccgccagcatcagctgcaagagcagccagagcctgctggacagcgacggcaagacctacctgaactggctgctgcagcggccaggccagagccccaagcggctgatctatctcgtcagcaagctggacagcggcgtgcccgaccggttcaccggcagcggcagcggcaccgacttcaccctgaagatcagccgggtggaggccgaggacctgggcgtgtactactgctggcagggcacccactttccccggaccttcggcggcggcaccaagctggagatcaagcgggccgacgccgccccaacagtcagcatctttccaccatcctccgagcagctcaccagcggcggcgccagcgtggtgtgcttcctgaacaacttctaccccaaggacatcaacgtgaagtggaagatcgacggcagcgagcggcagaacggcgtgctgaacagctggaccgaccaggacagcaaggacagcacctacagcatgagcagcaccctgaccctgaccaaggacgagtacgagcggcacaacagctacacctgcgaggccacccacaagaccagcaccagccccatcgtgaagagcttcaaccggaacgagtgctga

SEQ ID NO: 6 - DNA, 2168 bp, Artificial Sequence
  A synthetic oligonucleotide sequence
gaggagccaccatggagtggacctgggtgttcctgttcctgctgagcgtgaccgccggcgtgcactcccaagtccagctgcagcagagcggcgccgagctgatgaagcccggcgccagcgtgaagatcagctgcaaggccaccggctacaccttcagcaactactggatcgagtgggtgaagcagcggcccggccacggcctggagtggatcggcgagatcctgcccggcagcgacagcatcaagtacaacgagaacttcaagggcaaggccaccttcaccgccgacaccagcagcaacaccgcctacatgcagctgagcagcctgaccagcgaggacagcgccgtgtactactgcgcccggcggggcaactaccccgactactggggccagggcaccaccctgaccgtctccagcgccaagaccacaccacccagcgtgtacccactggctccaggcagcgctgcccagaccaacagcatggtgaccctgggctgcctggtgaagggctacttccccgagcccgtgaccgtgacctggaacagcggcagcctgagcagcggcgtgcacaccttcccagccgtgctccaaagcgacctgtacacactgagcagcagcgtgaccgtgcccagcagcacctggcccagcgagaccgtgacctgcaacgtggcccaccccgccagcagcaccaaggtggacaagaagatcgtgccccgggactgcggctgcaagccctgcatctgcaccgtgcccgaagtcagcagcgtgttcatcttcccacccaagcccaaggacgtgctgaccatcaccctgacacccaaagtcacctgcgtggtggtggacatcagcaaggacgaccccgaggtgcagttcagctggttcgtggacgacgtggaggtgcacaccgcccagacccagccccgggaggagcagttcaacagcaccttccggagcgtgagcgagctgcccatcatgcaccaggactggctgaacggcaaggagttcaagtgccgggtgaacagcgccgccttccccgcccccatcgagaagaccatcagcaagaccaagggacggcccaaggctccccaggtgtacaccatcccaccacccaaggagcagatggccaaggacaaagtcagcctgacctgcatgatcaccgacttcttccccgaggacatcaccgtggagtggcagtggaacggccagcccgccgagaactacaagaacacccagcccatcatggacaccgacggcagctacttcgtgtacagcaagctgaacgtgcagaagagcaactgggaggccggcaacaccttcacctgcagcgtgctgcacgagggcctgcacaaccatcacaccgagaagagcctgagccacagcccaggacggaagcgccgcgagggcaggggcagcctgctgacctgcggcgacgtggaggagaacccaggccccatgaagctgcccgtgcggctgctggtgctgatgttctggatccccgccagcagcagcgacgtggtgatgacccagaccccactgagcctgcccgtgagcctgggcgaccaggccagcatcagctgccggagcagccagagcctggtgcacagcaacggcaacacctacctgcactggtacctgcagaagcccggccagagccccaagctgctgatctacaaagtcagcaaccggttcagcggcgtgcccgaccggttcagcggcagcggcagcggcaccgacttcaccctgaagatcagccgggtggaggccgaggacctgggcgtgtacttctgcagccagagcacccacgtgcccctgaccttcggcgccggcaccaagctggagctgaagcgggccgacgccgctcccaccgtcagcatcttcccaccctcctccgagcagctgaccagcggaggcgccagcgtggtgtgcttcctgaacaacttctaccccaaggacatcaacgtgaagtggaagatcgacggcagcgagcggcagaacggcgtgctgaacagctggaccgaccaggacagcaaggacagcacctacagcatgagcagcaccctgaccctgaccaaggacgagtacgagcggcacaacagctacacctgcgaggccacccacaagaccagcaccagccccatcgtgaagagcttcaaccggaacgagtgctga